(19) United States
(12) Patent Application Publication
(10) Pub. No.: US 2005/0048661 A1
(43) Pub. Date: Mar. 3, 2005

(54) Methods and apparatus for analyzing materials

(76) Inventors: Jimmy L. Droit, Glendale, AZ (US); Glenn A. Johnson, Mesa, AZ (US); Patrick Lee Harvey, Scottsdale, AZ (US)

Correspondence Address: Law Offices of Daniel J. Noblitt, LLC;Suite 123, 3370 North Hayden Road, Box 258, Scottsdale, AZ, 85251, US

(21) Appl. No.: 10/648,147

(22) Filed: Aug. 25, 2003

(51) Int. Cl. G01N 33/00

(52) U.S. Cl. 436/155; 422/68.1

(57) ABSTRACT

A method and apparatus for testing materials according to various aspects of the present invention comprises an oven and a control system. The oven is configured to receive a test material for testing and expose the test material to heat to promote drying and/or ashing. The control system receives data, such as temperature data from the oven, and controls the temperature in the oven accordingly.

### FIELD OF THE INVENTION

The invention relates to methods and apparatus for analyzing characteristics of materials.

### BACKGROUND OF THE INVENTION

Industrial and commercial operations often test materials to determine their water content. A wide array of devices has been developed to perform water content tests.

Such devices may use various techniques for determining water content, such as loss-on-drying methods. Loss-on-drying methods generate data based on a test material's weight loss while drying in an oven. Oven techniques are effective, but require hours of time. Using a loss-on-drying moisture analyzer with a small oven chamber dramatically speeds the testing.

Other tests may also be performed on test samples, such as an ashing test for a test material's oxidizing components. An oven can be used to ash the material. In conducting the test, the temperature is typically slowly ramped up avoid an explosion, which may affect the test material and/or damage the test equipment. Once the desired ashing temperature is obtained, the sample continues to burn until the oxidizing components are exhausted. The material weight loss during the test corresponds to the amount of oxidizing components.

### SUMMARY OF THE INVENTION

A method and apparatus for testing materials according to various aspects of the present invention comprises an oven and a control system. The oven is configured to receive a test material for testing and expose the test material to heat to promote drying and/or ashing. The control system receives data, such as temperature data from the oven, and controls the temperature in the oven accordingly.

### BRIEF DESCRIPTION OF THE DRAWING

A more complete understanding of the present invention may be derived by referring to the detailed description when considered in connection with the following illustrative figures. In the following figures, like reference numbers refer to similar elements and steps.

FIG. 1 is a perspective view of a materials analysis system according to various aspects of the present invention;

FIG. 2 is a cross-sectional view of the materials analysis system;

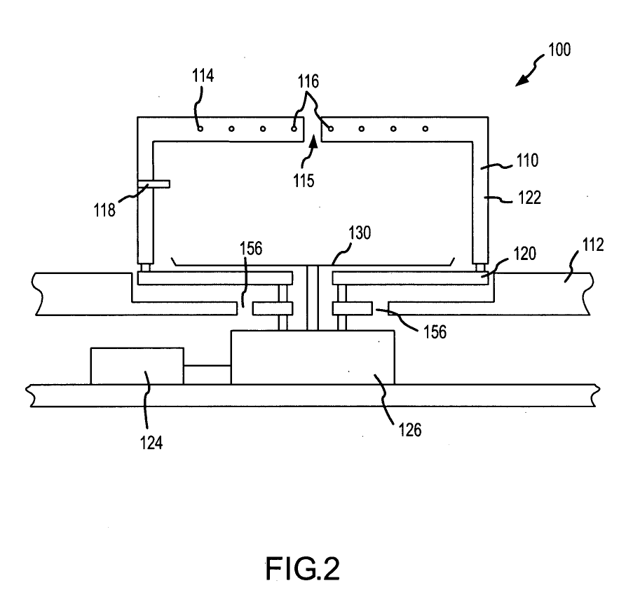

FIG. 3 is a block diagram of various elements of the materials analysis system;

FIG. 4 is a block diagram of a control system connected to a power source and a heating element;

FIG. 5 is a circuit diagram of a timing circuit;

FIG. 6 is a flow diagram of a loss-on-drying process;

FIG. 7 is a flow diagram of an ashing process; and

FIG. 8 is a flow diagram of a heating element control process.

Elements and steps in the figures are illustrated for simplicity and clarity and have not necessarily been rendered according to any particular sequence. For example, steps that may be performed concurrently or in different order are illustrated in the figures to help to improve understanding of embodiments of the present invention.

### DETAILED DESCRIPTION OF EXEMPLARY EMBODIMENTS

The present invention is described partly in terms of functional components and various processing steps. Such functional components may be realized by any number of components configured to perform the specified functions and achieve the various results. For example, the present invention may employ various elements, materials, heating elements, control systems, and the like, which may carry out a variety of functions. In addition, the present invention may be practiced in conjunction with any number of applications, environments, test materials, and control algorithms, and the systems described are merely exemplary applications for the invention. Further, the present invention may employ any number of conventional techniques for manufacturing, assembling, processing, and the like.

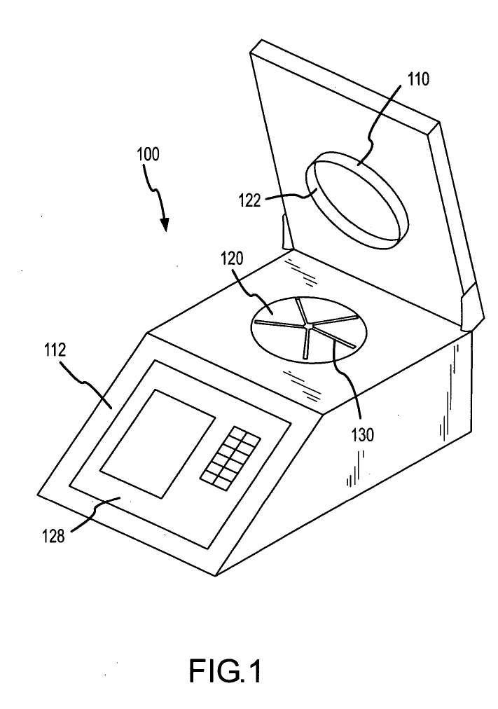

Referring now to FIG. 1 and, a method and apparatus for material analysis according to various aspects of the present invention comprises a materials analysis system 100 for analyzing characteristics of materials. The materials analysis system 100 may comprise, for example, an oven 110 and a base 112. The oven 110 heats test material in the oven 110. The base 112 supports the oven 110 and contains components to measure changes in the test materials upon exposure to heat.

The oven 110 may comprise any suitable system for heating the materials to be tested, such as a radiative oven or a microwave oven. The oven 110 may be configured in any suitable manner, however, to heat the test materials. For example, the oven 110 suitably comprises a convection oven having a housing 114 and a heating element 116. The housing 114 houses the test materials during heating, and the heating element 116 heats the test materials.

More particularly, the housing 114 may comprise any appropriate mechanism for maintaining the heat applied to the test materials. The housing 114 is also suitably configured to isolate heat generated in the oven 110 from the surrounding environment. For example, in the present embodiment, the housing 114 comprises an enclosure having a bottom portion 120 and a top portion 122. The bottom portion 120 and the top portion 122 are connected via a hinge so that the top portion 122 may be moved relative to the bottom portion 120 to allow access to the interior of the housing 114. The housing 114 may have one or more holes to vent moisture, smoke, and the like from within the housing 114, such as a hole 115 in the top of the top portion 122.

The housing 114 may comprise any suitable materials for maintaining heat within the oven 110. To retain heat within the housing 114 and inhibit transfer of heat to the exterior, the housing 114 suitably comprises a thermal insulator, such as a plastic, a vacuum, a ceramic, or multiple materials. In the present embodiment, the housing 114 comprises a ceramic or ceramic fiber material, which tends to retain heat within the housing 114 and withstand high temperatures. Consequently, the oven may be operated at elevated temperatures, such as in excess of 300° C. and up to about 600° C. or more.

The heating element 116 may comprise any suitable system for heating the test materials. For example, the heating element 116 suitably comprises a radiative heating element that radiates heat. Alternatively, the heating element 116 may comprise a microwave system for generating microwaves to heat the test materials or other appropriate mechanism for heating the test materials. In the present embodiment, the heating element 116 comprises a resistive heater, such as a nichrome wire, coil, or foil. The heating element 116 may embedded in the housing 114, such as in the walls, roof, or floor of the housing 114. In the present embodiment, the heating element 116 is embedded in the roof of the housing 114.

The oven 110 may also include any other suitable components for operating the materials analysis system 100. For example, the housing 114 suitably includes a temperature sensor 118. The temperature sensor 118 may comprise any suitable sensor and be configured in any appropriate manner to measure the temperature within the housing 114. For example, the temperature sensor 118 may comprise a conventional high-precision and -accuracy resistance temperature device. In the present embodiment, the temperature sensor 118 is at least partially exposed within the interior of the housing 114.

In the present embodiment, the oven 110 is supported by the base 112, which also encloses various other components of the materials analysis system 100. The base 112 may comprise any suitable mechanism for supporting the oven 110 and enclosing various components, such as a conventional metal enclosure. The base 112 suitably includes a fan (not shown) for introducing air into the base 112 to cool the interior of the base 112 and the components inside. In addition, the base of the present embodiment includes one or more holes 156 formed in the top of the base 112 under the bottom portion 120 of the oven 110. Air driven by the fan escapes through the holes 156 to remove moisture from underneath the bottom portion 120, for example from seepage.

The base 112 may contain components for the operation of the materials analysis system 100, such as a control system 124, a mass measuring system 126, and an interface 128. The control system 124 controls various operational aspects of the materials analysis system 100. The mass measuring system 126 measures the mass of the test materials, and the interface 128 facilitates transfer of information to and from the materials analysis system 100.

The mass measuring system 126 may comprise any suitable system for determining the mass of the test materials. In the present embodiment, the mass measuring system 126 comprises a high-accuracy weighing mechanism, such as a precision electronic balance with a resolution of about 0.0001 gram. The mass measuring system 126 also suitably has a full scale range sufficient for the anticipated test materials, such as 100 grams.

The mass measuring system 126 maybe configured to measure the mass of the test material in any suitable manner, such as by measuring the weight of the test materials directly or indirectly. In the present embodiment, the mass measuring system 126 is connected to a pan support 130 within the oven 110 so that the mass measuring system 126 may measure the weight of the test materials in the oven 110. The pan support 130 supports a pan on which the test material rests. In the present embodiment, the pan support 130 has more than three prongs, such as five prongs, so that if the pan softens during testing, the pan is inhibited from touching the floor of the oven 110 and affecting the mass measurement.

The interface 128 is suitably configured to transfer information to and from the materials analysis system 100, such as to and from a human operator or another machine. In the present embodiment, the interface 128 includes both a human interface and a machine interface. The human interface may comprise any suitable system for exchanging information with a human. For example, in the present embodiment, the human interface comprises a display, such as a liquid crystal display, a keypad for typing information into the materials analysis system 100, and a speaker. The machine interface may comprise any suitable system for communicating with other machines. In the present embodiment, the machine interface comprises one or more connections for other machines, such as a printer port, a network connection such as an Ethernet port, an RS-232 port, a serial port, and/or any other suitable connection.

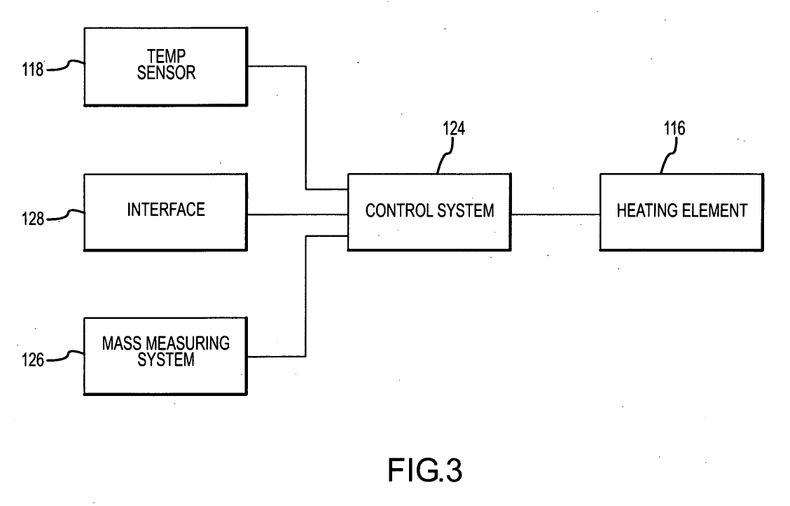

The control system 124 controls the operations of the materials analysis system 100. The control system 124 may comprise any suitable system, such as a controller 136 operating in conjunction with a memory 134. In addition, the control system 124 may be connected with various other components of the materials analysis system 100. For example, referring to FIG. 3, the control system 124 is suitably connected to the temperature sensor 118, the interface 128, the mass measuring system 126, and the heating element 116. The control system 124 may monitor the temperature within the oven 110 via the temperature sensor 118 and control the heating element 116 to control the temperature in accordance with control criteria received via the interface 128, stored in the memory 134, in accordance with a control algorithm, or the like. The control system 124 may also control the materials analysis system 100 to achieve various tasks, such as setup, memory accesses, cleaning, and calibration, as well as testing processes, such as loss-on-drying tests and ashing tests.

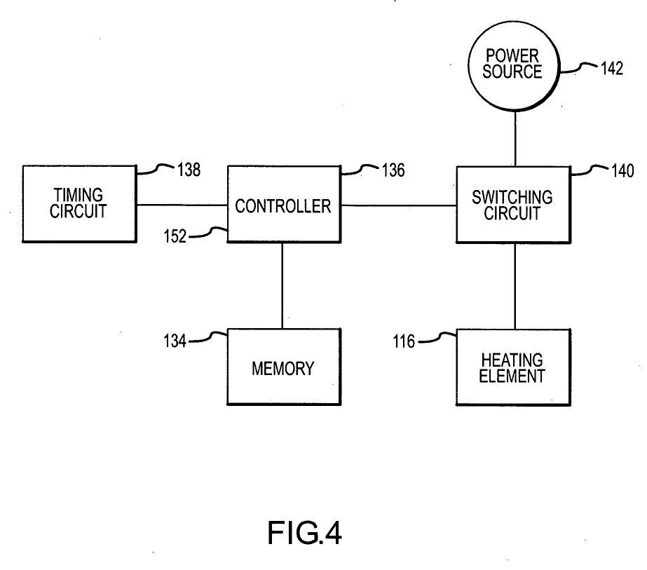

For example, referring to FIG. 4, a control system 124 according to various aspects of the present invention comprises a controller 136, a timing circuit 138, and a switching circuit 140. The switching circuit 140 receives signals from the controller 136 and the timing circuit 138 to activate and deactivate the heating element 116. The timing circuit 138 synchronizes the activation of the heating element 116 with a power source 142. The controller 136 controls the heating element 116 according to one or more parameters, such as the temperature signal received from the temperature sensor 118, a rate at which the test material weight changes, a control algorithm, and/or input parameters received from, for example, the memory 134 and/or the interface 128.

More particularly, the switching circuit 140 may comprise any suitable mechanism for controlling the heating element 116. For example, the switching circuit 140 may comprise an electrically operated switch, such as a relay or triac. In the present embodiment, the switching circuit 140 comprises a zero crossing triac configured to selectively connect the heating element 116 to the power source 142. The gate of the triac is suitably connected to an output of the controller 136.

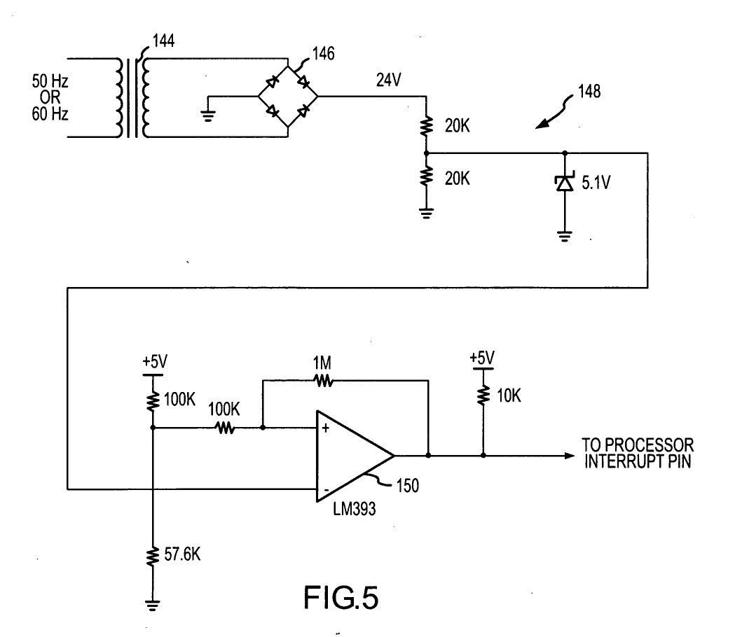

The timing circuit 138 may be configured in any suitable manner to synchronize the operation of the heating element 116 with the power source 142. Referring to FIG. 5, in the present embodiment, the timing circuit 138 comprises a transformer 144, a rectifier 146, an amplitude adjustment circuit 148, and a comparison circuit 150. The transformer 144 delivers a signal at the same frequency as the power source 142 but at a lower voltage, such as 24 volts. The rectifier 146 fully rectifies the transformer 144 signal to generate a rectified signal at twice the line frequency. The amplitude adjustment circuit 148 adjusts the amplitude of the rectified signal to a desired maximum level, such as a five-volt logic level, for example via a voltage divider circuit and a zener diode having a 5.1 volt breakdown voltage. The comparison circuit 150 compares the adjusted signal to a threshold voltage, such as 2.5 volts, and has a 5-volt pull-up transistor. Consequently, the timing circuit 138 generates a timing signal at twice the line frequency at a selected voltage level.

Referring again to FIG. 4, the controller 136 controls the operation of the heating element 116. The controller 136 may comprise any suitable system for controlling the operation of the heating element 116, such as a hardwired control system or a programmable control system, for example a programmable logic array or a microprocessor. In the present embodiment, the controller 136 comprises a microprocessor 152 operating in conjunction with the memory 134. The microprocessor 152 receives one or more signals and controls the materials analysis system 100 according to the received signals and a control algorithm.

For example, the microprocessor 152 suitably receives the timing signal, such as via an interrupt input to initiate an interrupt routine that activates or deactivates the switching circuit 140 in accordance with the control algorithm. The timing signal may be used to synchronize the operation of the heating element 116 with the power source 142. The microprocessor 152 also receives the temperature signal from the temperature sensor 118. The microprocessor 152 may then activate and deactivate the heating element 116 to control the temperature within the oven 110.

The control system 124 may control the temperature according to any suitable process or algorithm. In the present embodiment, the control system 124 is configured to control the temperature using different processes to perform different operations, such as a loss-on-drying analysis, an ashing analysis, and a self-cleaning operation.

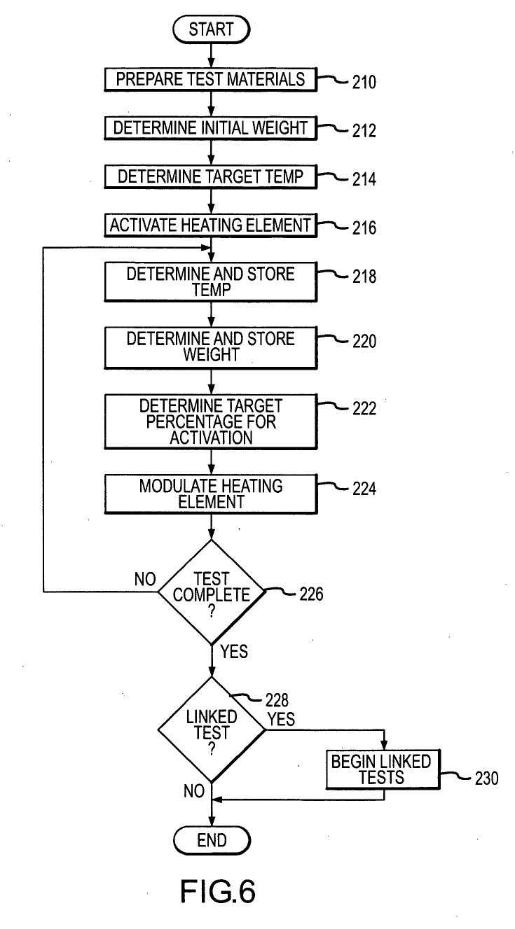

A loss-on-drying process suitably measures the difference in the weight of the test materials as they are subjected to heat, causing volatiles in the test materials, such as water, alcohol, and the like, to vaporize and exit the test material. The loss-on-drying process may be performed, however, in any suitable manner. For example, referring to FIG. 6, a loss-on-drying process 200 according to various aspects of the present invention comprises preparing the test materials (210), placing the test materials on a pan having an established weight, and placing the pan on the pan support. The mass measuring system 126 determines the initial weight and stores it in memory (212).

In the present embodiment, the control system 124 is configured to control the temperature within the oven 110 according to a target temperature. The target temperature may be determined by any suitable process, such as via an input from the interface 128 or retrieved from the memory 134. In the present embodiment, the control system 124 determines the target temperature (214). The target temperature may be selected or calculated in any suitable manner, such as manually selecting a setpoint temperature or calculating a temperature to generate selected data. The control system 124 activates the heating element 116 (216) to begin heating the oven 110. As the test begins, the temperature in the oven 110 (218) and weight of the test materials (220) are monitored at selected intervals. In addition, the control system 124 suitably calculates the rate of weight loss for the test material at intervals, such as five-second intervals.

The control system 124 controls the heating element 116 to achieve the desired temperature. Any suitable process or algorithm may be used to establish the desired temperature. For example, the control system of the present embodiment 124 uses a feedback process, such as a proportional-integral-differential (PID) algorithm, to control the oven temperature by determining a target percentage of the cycle time during which the heating element 116 should be activated (222).

The control system 124 may use different calculation processes or conditions at various points in the test process. For example, the control system may use different algorithms, different constants for the PID algorithm, or otherwise vary the control parameters within different temperature ranges. In the present embodiment, the possible temperatures are divided into multiple temperature zones, such as seven zones, that are accorded different sets of constants. For example, at the lowest temperature range, such as below 55° C., the proportional constant may be about four times higher than for the highest temperature range, such as above 450° C. The differential and integral constants may also be varied for the different zones. By modifying the control process according to the current temperature in the oven 110, the control system 124 may inhibit overshooting the target temperature while retaining the ability to rapidly ramp up to a target temperature.

The control system 124 suitably controls the heating element 116 according to the target calculated percentage. For example, the control system 124 may adjust the current through the heating element or the duration of activation of the heating element 116. In the present embodiment, the control system 124 activates and deactivates the triac according to the target calculated percentage and the timing circuit signal.

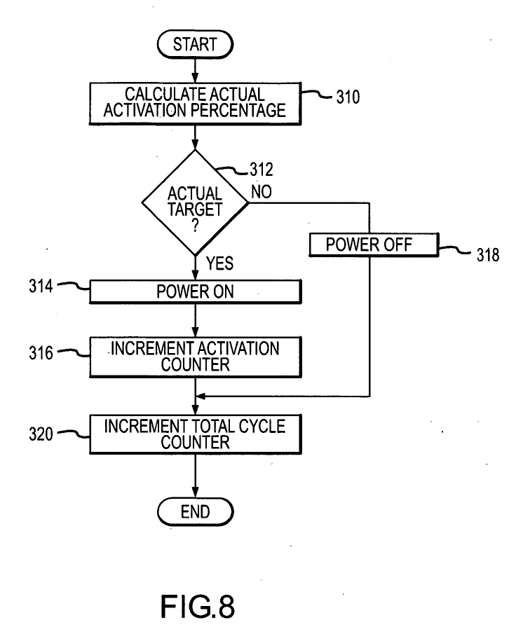

The control system 124 suitably modulates power provided to the heating element 116 using pulse width modulation process having a variable period (224). More particularly, referring to FIG. 8, the control system 124 of the present embodiment calculates an actual activation percentage of time or number of cycles over a selected period, for example the elapsed time since the target calculated percentage was last changed, during which the heating element 116 has been activated (310). The control system 124 may calculate the actual activation percentage and compare it to the target calculated percentage at selected intervals or in response to a signal (312). In the present embodiment, the control system 124 performs the calculation and comparison in response to the timing signal, or at double the line frequency.

If the actual activation percentage is lower than the target calculated percentage, the control system 124 activates or maintains the activation of the heating element 116 (314). The control system 124 may also increment a counter indicating the number of cycles or time for which the heating element 116 has been activated (316). If the actual activation percentage is not lower than the target calculated percentage, the control system 124 deactivates or maintains the deactivation of the heating element 116 (318). The control system 124 then increments a counter indicating the total number of relevant cycles (320). The total cycle counter is suitably reset upon a change in the target calculated percentage or at the expiration of a timer, such as a 30-second timer, to avoid overflow.

As a result, the control system 124 adjusts the activation duration of the heating element 116 as in a pulse width modulation scheme. Because the duration in which the heating element is activated and deactivated is not based on a constant period, however, the period effectively varies to more closely approximate the desired target calculated percentage. For exanple, in the present embodiment, the signal to the switching circuit 140 is updated every half cycle, or every 8.33 milliseconds for 60 Hz line frequency. The control system is suitably configured to control the heating element 116 power to a selected precision, such as 0.1%. To adjust the power to 50%, the control system would provide power at 8.33 ms ON and 8.33 ms OFF repeated. Correspondingly, 0.1% power would be 8.33 ms ON and 8.3247 seconds OFF repeated. 33.3% power may be implemented by turning the power ON for 8.33 ms and OFF for 16.67 ms, such that the period is 25 milliseconds. Thus, the frequency in this example may change anywhere between 60 Hz and 0.12 Hz, or the period may change from 16.67 milliseconds to 8.33 seconds and anywhere in between. Thus, in the present embodiment, the period of the signal to the switching circuit 140 may be as short as the period of the line signal.

The control system 124 suitably repeats the process (226) until a selected end criterion is met. The end criterion may comprise any appropriate criteria for ending the test, such as expiration of a time limit, arriving at a selected loss rate, or upon a predicted result. If the test is completed, the results are suitably stored. In addition, the control system 124 may calculate additional values based on the test results, such as standard deviations or other values.

The control system 124 may then determine whether another test should be conducted consecutively on the test materials (228), such as another loss-on-drying test at higher temperatures, or a different test, such as an ashing test. Conducting such tests consecutively without opening the oven 110 or remove the test materials inhibits absorption of water or other contamination from outside the materials analysis system 100. For example, consecutive loss-on-drying tests at stepped temperatures may help to assess amounts of free water and bound water, respectively, in a particular material. In addition, the materials analysis system 100 of the present invention is configured to proceed, if desired, from completion of the loss-on-drying process to the ashing process using the same test material. If another test is to be performed consecutively, the control system 124 initiates the test (230). If not, the test process is terminated and the materials analysis system 100 returns to an idle mode.

The control system 124 may also be configured to perform different tests. For example, the present control system 124 may be configured to perform an ashing test to facilitate the oxidation of components in the test materials without igniting the materials. In the present embodiment, the materials analysis system 100 is configured to perform that ashing test at an enhanced rate.

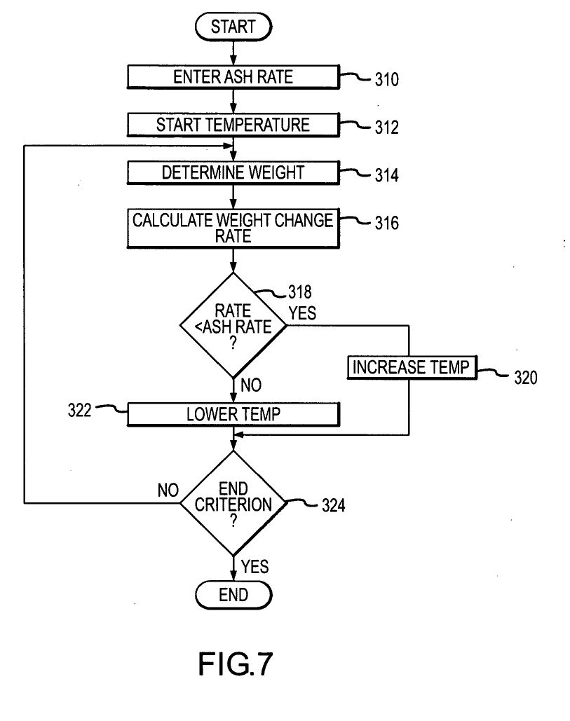

For example, referring to FIG. 7, the ash rate for the relevant material is suitably entered into the control system 124 (310), for example via the interface 128. When the materials are in the oven 110, the control system 124 adjusts the heating element 116 to bring the temperature to a starting temperature such as 100° C. (312). When nearing the starting temperature, the control system 124 measures the weight of the test materials (314) and calculates a weight change rate (316). If the weight change rate is lower than the ash rate (318), the control system 124 increases the temperature (320). Any time the weight change rate is higher than the ash rate, the control system reduces the temperature (322). The process repeats until an end criterion is satisfied (324), such as a particular weight change rate signifying that substantially all possible components of the test materials have been oxidized. Consequently, the weight change rate may be maintained below the ash rate to inhibit ignition.

The control system 124 may also perform a self-cleaning operation. In particular, the control system 124 may heat the oven 110 to a relatively high temperature, such as about 550° C., for a selected period, such as about 45 minutes. Elevating the temperature for an extended period tends to burn off extra materials that may be lodged within the oven 110. Upon expiration of the relevant period, the control system 124 suitably returns the materials analysis system 100 to its idle condition.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The present invention has been described above with reference to a preferred embodiment. However, changes and modifications may be made to the preferred embodiment without departing from the scope of the present invention. These and other changes or modifications are intended to be included within the scope of the present invention.

1. A materials analysis system for testing a test material, including:
an oven, including:
a housing comprising at least one of a ceramic or a ceramic fiber; and
a heating element in communication with the housing and configured to transfer heat to an interior of the housing;
a control system configured to control the heating element; and
a mass measuring system connected to the control system and having a test
material support associated with the oven housing.

2. A materials analysis system according to claim 1, wherein the control system is configured to control the heating element according to a first criterion when the interior of the housing is at a first temperature and according to a second criterion when the interior of the housing is at a second temperature.

3. A materials analysis system according to claim 2, wherein the control system is configured to control the heating element according to a feedback process, wherein the first criterion is a first constant used in the feedback process and the second criterion is a second constant used in the feedback process.

4. A materials analysis system according to claim 3, wherein the feedback process includes a PID algorithm.

5. A materials analysis system according to claim 1, wherein the control system is configured to control the heating element according to:
a weight change rate for the test material based on a signal from the mass measuring system; and
a predetermined ashing rate.

6. A materials analysis system according to claim 1, wherein the control system is configured to consecutively perform a loss-on-drying process to the test material and an ashing test to the test material.

7. A materials analysis system according to claim 1, wherein the control system is configured to control the heating element using a modulated signal having a variable period.

8. A materials analysis system according to claim 1, wherein the control system is configured to execute a self-cleaning process.

9. A materials analysis system according to claim 1, wherein the heating element comprises a radiative heating element.

10. A materials analysis system according to claim 1, wherein the test material support includes at least four prongs.

11. A materials analysis system for analyzing a test material, comprising:
an oven, including:
a housing including a ceramic; and
a radiative heating element;
a control system connected to the heating element and configured to control the heating element to execute at least one of a loss-on-drying process and an ashing process on the test material.

12. A materials analysis system according to claim 11, further comprising a mass measuring system connected to the control system and configured to weigh the test material in the oven.

13. A materials analysis system according to claim 11, further comprising a temperature sensor at least partially disposed within the oven housing and connected to the control system.

14. A materials analysis system according to claim 11, wherein the control system:
includes a memory configured to store multiple sets of constants, and
is configured to control the heating element according to different sets of constants retrieved from the memory when the interior of the housing is at different temperatures.

15. A materials analysis system according to claim 14, wherein the control system is configured to control the heating element according to a feedback process, wherein the different sets of constants are used in the feedback process.

16. A materials analysis system according to claim 15, wherein the feedback process includes a PID algorithm.

17. A materials analysis system according to claim 11, wherein the control system is configured to control the heating element to execute the ashing process according to:
a weight change rate for the test material calculated by the control system based on a weight signal from the mass measuring system; and
a predetermined ashing rate associated with the test material.

18. A materials analysis system according to claim 11, wherein the control system is configured to consecutively perform the loss-on-drying process to the test material and the ashing test to the test material.

19. A materials analysis system according to claim 11, wherein the control system is configured to control the heating element using a frequency modulated signal.

20. A materials analysis system according to claim 11, wherein the control system is configured to control the heating element according to a target activation value and an actual activation value.

21. A materials analysis system according to claim 11, wherein the control system is configured to execute a self-cleaning process.

22. A materials analysis system according to claim 11, wherein the test material support includes at least four prongs.

23. A method of testing a test material, comprising:
determining an initial weight of the test material;
exposing the test material to a temperature in excess of 300° C. in an oven;
measuring a change of the temperature in the oven;
measuring a change of weight of the test material; and
controlling a heating element for the oven according to at least one of the change of temperature in the oven and the change of weight of the test material.

24. A method of testing a test material according to claim 23, wherein controlling the heating element includes frequency modulating a power provided to the heating element.

25. A method of testing a test material according to claim 23, wherein controlling the heating element includes controlling the heating element in conjunction with a PID algorithm.

26. A method of testing a test material according to claim 25, wherein controlling the heating element includes:
using a first set of constants in the PID algorithm when the temperature in the oven is within a first range; and
using a second set of constants in the PID algorithm when the temperature in the oven is within a second range.

27. A method of testing a test material according to claim 23, wherein controlling the heating element includes controlling the heating element in conjunction with a preselected ash rate for the test material.

28. A method of testing a test material according to claim 23, wherein controlling the heating element includes:
determining a target activation value for the heating element;
determining an actual activation value for the heating element; and
deactivating the heating element according to a relationship between the target activation value and the actual activation value.
